(19) United States
(12) Patent Application Publication
(10) Pub. No.: US 2008/0281277 A1
(43) Pub. Date: Nov. 13, 2008

(54) Device for Sealing an Opening in a Human or Animal Eye

(75) Inventor: Reinhard Thyzel, Heroldsberg (DE)

Correspondence Address: OSHA LIANG L.L.P., 1221 MCKINNEY STREET, SUITE 2800, HOUSTON, TX, 77010, US

(73) Assignee: Reinhard Thyzel, Heroldsberg (DE)

(21) Appl. No.: 11/884,895

(22) PCT Filed: Jan. 26, 2006
(86) PCT No.: PCT/EP2006/000653

§ 371 Date: Apr. 11, 2008

(30) Foreign Application Priority Data
Feb. 22, 2005 (DE) 10 2005 008 235.1

(51) Int. Cl. A61F 9/007

(52) U.S. Cl. 604/256

(57) ABSTRACT

The apparatus for sealing an opening (18) in a human or animal eye (10) comprises at least one sealing element (2) a) with a passage (25, 26) surrounded by a wall, b) and with at least one sealing region (20) which can be positioned or is positioned on an outer surface of the eye tissue surrounding the opening with a sealing surface completely surrounding the opening, c) in which an operating instrument (3) can be passed or is passed into the eye interior (15, 17) through the passage in the sealing element and through the opening.

The invention relates to a device for sealing an opening in a human or animal eye.

The human eye or the eyeball comprises an outer membrane which is divided into the cornea, sclera and sclerotic coat and which encloses an intraocular space which, viewed in the direction of incidence of light, can be divided into an anterior chamber, a posterior chamber, and a vitreous chamber, in which the vitreous body is arranged. Arranged between the anterior chamber and the vitreous body is the lens, which is suspended by the zonular fibers on the ciliary muscle system formed on the sclerotic coat and can thus be modified in terms of its convexity and, consequently, its optical focal distance (accommodation). The vitreous body is surrounded by the retina, which is connected to the brain via the optic nerve. The cornea forms the front transparent area of the outer membrane of the eye and forms an optical imaging system in combination with the transparent liquid located in the eye chamber, mainly composed of water, and with the lens and the vitreous body. The image formed on the retina by means of this optical imaging system is taken up by the retina and forwarded to the brain via the optic nerve.

In the eye interior, there is an ocular pressure or intraocular pressure that is greater than the external pressure or atmospheric pressure (normal pressure). In particular, therefore, the liquid in the chamber of the eye is at a higher internal pressure.

In opthalmology, various surgical procedures are known in which instruments are used to perform surgical interventions in the eye interior. In these invasive intraocular procedures, at least one opening is created in the outer membrane of the eye, through which opening an instrument is inserted into the eye interior.

This opening is not pressure-tight, and this may possibly result in leaking of the aqueous humor. A drop in the intraocular pressure should be avoided, however. For this reason, flushing or irrigation liquid is introduced into the eye via the operating instrument itself, or via a separate second instrument, in order to be able to continuously compensate for the loss of pressure during the operation.

However, the resulting flow of liquid and the different pressures in most cases cause tissue damage or cell damage, with the result that the exchange of liquid or, in other words, the volumetric flow or throughput of the liquid during the operation should be kept as low as possible.

One intraocular surgical measure that is often performed involves replacement of a natural lens with an artificial (synthetic) lens (intraocular lens), which generally is made of a polymer material transparent in the visible spectrum, in particular acrylic glass (PMMA) or silicone (siloxane elastomer). In this surgical procedure, the natural lens is removed (explanted) from its capsular bag (capsula lentis), and an intraocular lens is then introduced (implanted) into the remaining capsular bag. In practice, the natural lens is explanted by destroying and ablating the lens tissue (phacolysis), generally by phacoemulsification, in which the lens is emulsified (liquefied) by means of ultrasound, or by means of shock waves generated with laser light (photolysis), and is suctioned off. The use of foldable lenses or injectable lenses permits a reduction in the size of the surgical incision to in practice only 2 mm, or even smaller. Replacement of the natural lens by a synthetic intraocular lens is presently performed primarily for removing a cataract. However, other applications are also possible, for example the implantation of an intraocular lens for adaptation or correction of the optical focal distance, for example in cases of nearsightedness (myopia) or farsightedness (hyperopia), or following accidents or injuries to the lens in which the capsular bag itself is not damaged irreparably.

U.S. Pat. No. 5,324,281 A discloses a surgical instrument in the form of a needle for destroying tissue, intended for the photolytic removal of cataracts. This known instrument has a needle, and also a laser fiber and a suction channel that each extend longitudinally and within the interior of the needle to the free end thereof. Arranged at the free end of the needle, there is a target made of titanium (Ti) set at a distance from the free end of the laser fiber, and the laser fiber and the target are adapted to one another in such a way that the laser light from this fiber strikes the target. Moreover, the free end of the needle is provided with a tissue-receiving opening which is arranged obliquely and in a laterally offset position and into which the suction channel opens, and which is arranged directly adjacent to the target and to the space between the laser fiber end and the target. An underpressure (vacuum) is generated in the suction channel by means of a suction pump and is used to suction the tissue that is to be destroyed onto the tissue-receiving opening. When the tissue now lies on the tissue-receiving opening by means of the underpressure, the target is bombarded with laser pulses from the laser fiber, the laser pulses having sufficient energy to create an optical breach on the surface of the target material. This generates a shock wave that strikes the tissue located at the tissue-receiving opening and tears this tissue into small pieces that are then suctioned through the suction channel. The laser pulses have in particular a pulse duration of 8 ns and a pulse repetition rate of 20 pulses per second and are preferably generated with a neodymium-YAG laser with a wavelength of 1064 nm. Moreover, the needle can additionally be provided with a longitudinally extending irrigation channel for conveying irrigation liquid through a laterally arranged outlet opening.

U.S. Pat. No. 5,906,611 A discloses a development of the instrument known from U.S. Pat. No. 5,324,282 A, in which development the target is specially designed in a stepped form. Using a neodymium-YAG laser, pulses can be generated with pulse repetition rates of between 2 and 50 pulses per second and pulse energies of between 2 and 15 mJ. The pulse duration can be set between 8 and 12 ns. The pulse repetition rate is preferably set between 2 and 6 pulses per second and the pulse energy between 6 and 10 mJ. Between 200 and 800 pulses or shots are used for a cataract operation.

A laser handpiece with a similar structure to that disclosed in U.S. Pat. No. 5,324,282 A and U.S. Pat. No. 5,906,611 A, and with a digital control and supply unit comprising a laser for the laser pulses and a venturi pump for suctioning of the tissue parts, has already been available for some years under the name “Lyla/Pharo” from the company A. R. C. Laser GmbH and has been used successfully in a large number of operations. The laser handpiece is used for suctioning, and the irrigation with an electrolytic irrigation solution or flushing solution (BSS) is performed via a second instrument, in a bimanual technique. As regards the actual eye operation using this known device, different operating techniques are employed.

In one operation using the laser handpieces described above, the needle is inserted into the incision on the eye, whereupon the tissue tightly surrounds the needle and thus seals the needle against the inner edge surface of the outer membrane surrounding the opening.

In phacolysis with ultrasound, use is made of ultrasound instruments with an ultrasound needle that is set in axial oscillations by a piezoelectric drive and that is inserted through the incision and into the eye. This ultrasound needle vibrates with an ultrasound frequency, for example in the kilohertz range, for example 40 kHz. Such an ultrasonic phacoemulsification system from A. R. C. Laser GmbH is known under the name “Pharo”.

Because of the high mechanical energy, sealing of the ultrasound needle directly on the surrounding tissue of the outer membrane of the eye at the incision is not possible, since the membrane tissue would be destroyed or damaged upon contact with the ultrasound needle (thermal tissue damage, burning). In order to avoid contact or friction of the ultrasound needle on the membrane tissue in ultrasound phacolysis, the incision is therefore generally made larger than in the case of laser needles (typically 2.6 mm to 3.2 mm in ultrasound and typically 1.4 mm with laser).

At the same time, a sleeve made of a soft elastic and vibration-damping material, for example a silicone (siloxane rubber), is placed around the ultrasound needle, completely surrounds the needle and protects the outer membrane tissue edge around the incision from the vibrating needle. Between the needle and the sleeve, irrigation liquid is guided through the incision into the eye interior. The sleeve is pressurized and inflated outward and thus additionally seals the opening with respect to the tissue bearing laterally on the sleeve. In this sealing arrangement, the sleeve bears with its circumference on the side surfaces of the membrane that surround the opening, such that the length of the seal corresponds to the thickness of the outer membrane at this location. However, leaking of aqueous humor between the sleeve and tissue edge cannot be completely avoided in practice, one of the reasons being that the movements made by the operating surgeon result in spaces being continually formed between the outer membrane of the eye and the sleeve.

During extraction of the lens, the irrigation liquid should ideally only replace the volume of the suctioned lens tissue and compensate for the underpressure arising during this suctioning. This is not achieved in practice, because of the described lack of leaktightness at the openings. The underpressure during suctioning of tissue, for example in lens extraction, is typically from 700 to 800 mbar, that is to say 200 to 300 mbar below normal pressure or atmospheric pressure. This difference in pressure is comparatively high for the eye and necessitates a high degree of tightness or sealing of the opening on the eye.

To maintain sufficient stability of the chamber of the eye and a sufficient intraocular pressure, the volume of the liquid in the eye is therefore readjusted during an eye operation, since controlling the pressure in the liquid delivery system and in the hoses permits very rapid adjustment of the pressure in the eye by delivery of liquid or adjustment of the volumetric flow to the desired value. This necessitates quite considerable outlay in terms of control technology.

Another known intraocular surgical procedure is vitrectomy, in which the vitreous body is partly removed by means of a cutting instrument that is inserted through an opening in the eye. In this operation, a particularly high degree of sealing of the opening in the eye is required, or a low infusion pressure (typically 15 to 20 mbar and atmospheric pressure) is required, in order not to damage the retina. The size of the incision is typically 0.6 mm in this operation.

The object of the invention is now to make available a novel device for sealing an opening in a human or animal eye, in which the stated disadvantages of the prior art are at least partially rectified or completely avoided.

According to the invention, this object is achieved by the features of patent claim 1.

The invention is based on a concept whereby the opening made in the eye in opthalmological invasive procedures is not sealed off within the opening, but instead on the outer surface (or outwardly directed surface) of the eye tissue located around the opening. As tests have shown, this results in much better sealing, with considerably less loss of pressure and of liquid. This sealing is much less dependent on the shape and size of the opening than in the prior art and permits excellent sealing even of quite long incisions and also of large or small incisions (microincisions). Whereas the available sealing surface for sealing an eye opening in the prior art is limited by the thickness of the eye tissue at this location, and in addition the sealing is very sensitive to movements of the operating instrument relative to the opening, the sealing surface according to the invention can be adjusted in size and shape within wide limits and can also be placed at a distance from the opening, such that changes in the shape or size of the opening do not affect the sealing action.

In an operation for extraction of eye tissue, in particular lens tissue, the suction pressure or the volumetric flow needed for suction can be considerably increased and, consequently, the proportion of the aspiration during tissue removal or extraction can be set much higher, for example at 70%, and the proportion of the tissue destruction by the energy input by laser pulses or ultrasonic oscillations can be set considerably lower. The greater suction effect permits the aspiration of larger parts of tissue, such that the tissue does not have to be reduced into such small pieces before being suctioned off. Moreover, by virtue of the greater leaktightness and the practically closed pressure-tight system or irrigation/aspiration circuit, the flow rate and the required volumetric flow is smaller and the irrigation is easier to control. Particularly in the case of ultrasonic instruments too, the operation can be performed more gently, with less tendency to edema and contusions, and more safely, with reduced risk of infection, and burning of the eye tissue can be largely avoided.

Advantageous embodiments and developments of the device according to the invention are set forth in the claims dependent on claim 1.

The invention is explained in more detail below on the basis of illustrative embodiments. Reference is also made to the drawings listed below, in which:

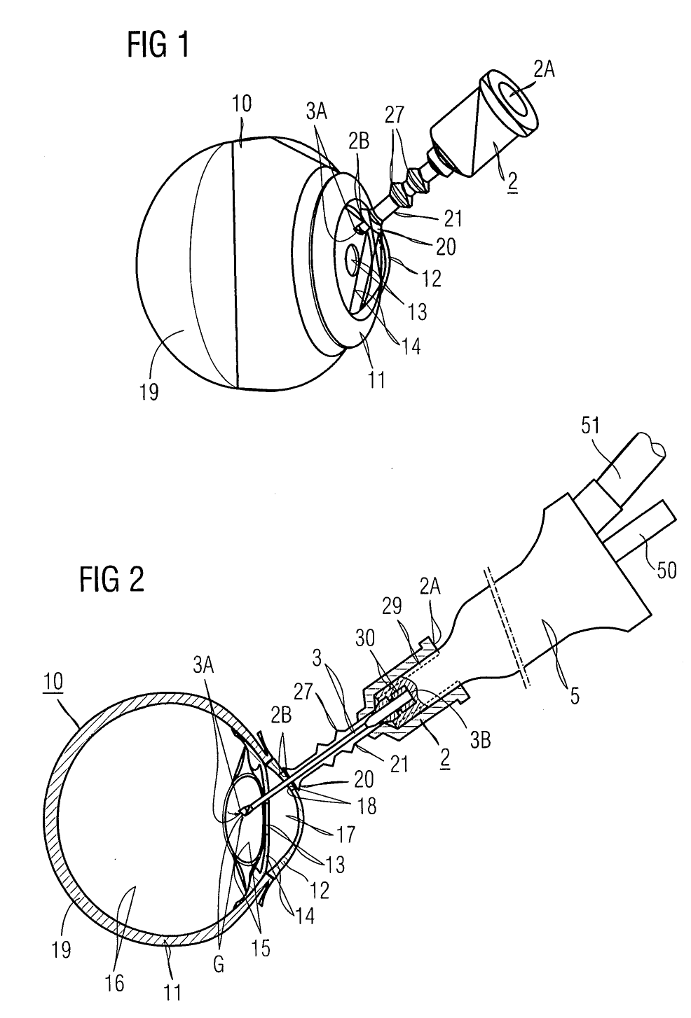

FIG. 1 shows a perspective view of a sealing element with an operating instrument received in it during a surgical intervention on an eye,

FIG. 2 shows the arrangement according to FIG. 1 in a cross-sectional view,

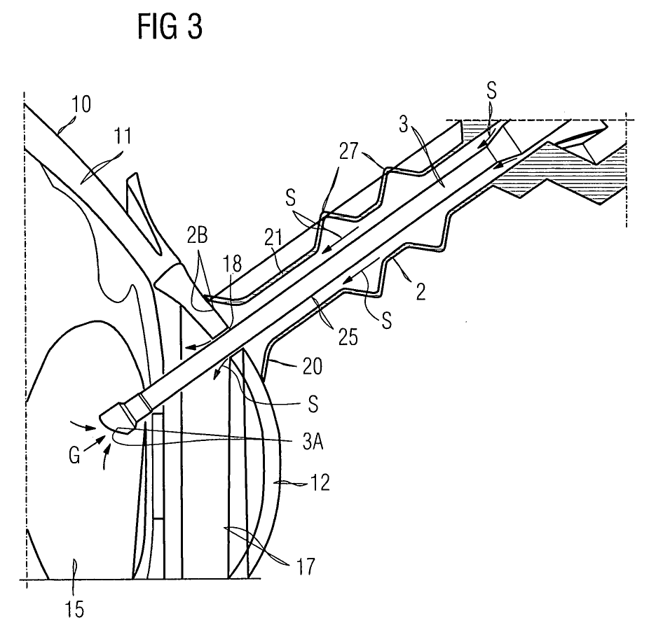

FIG. 3 shows the arrangement according to FIG. 2 in an enlarged detail,

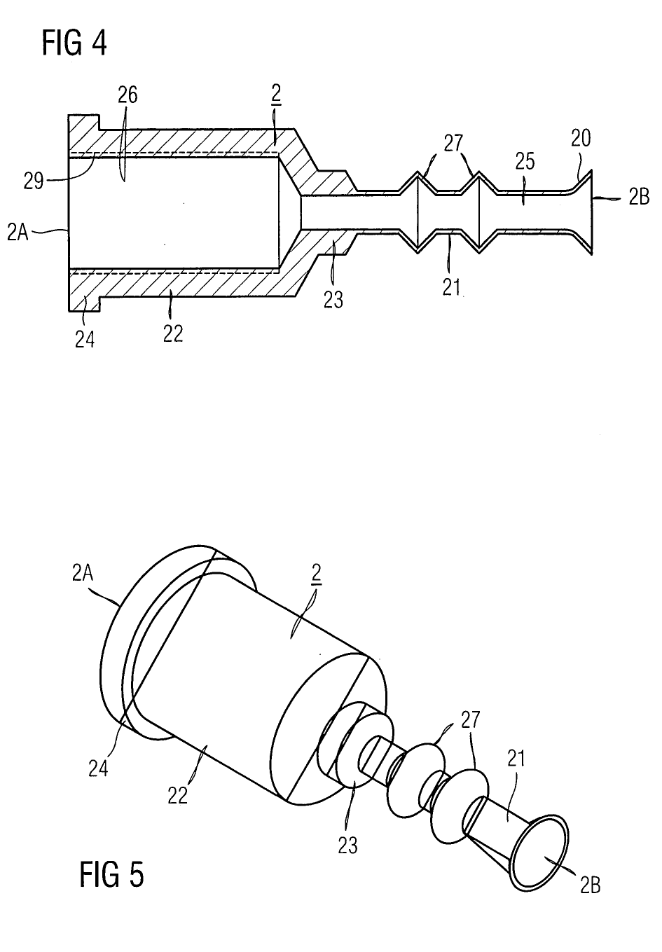

FIG. 4 shows the sealing element according to FIGS. 1 to 3 on its own, in a cross-sectional view,

FIG. 5 shows the sealing element according to FIG. 4 in a perspective view,

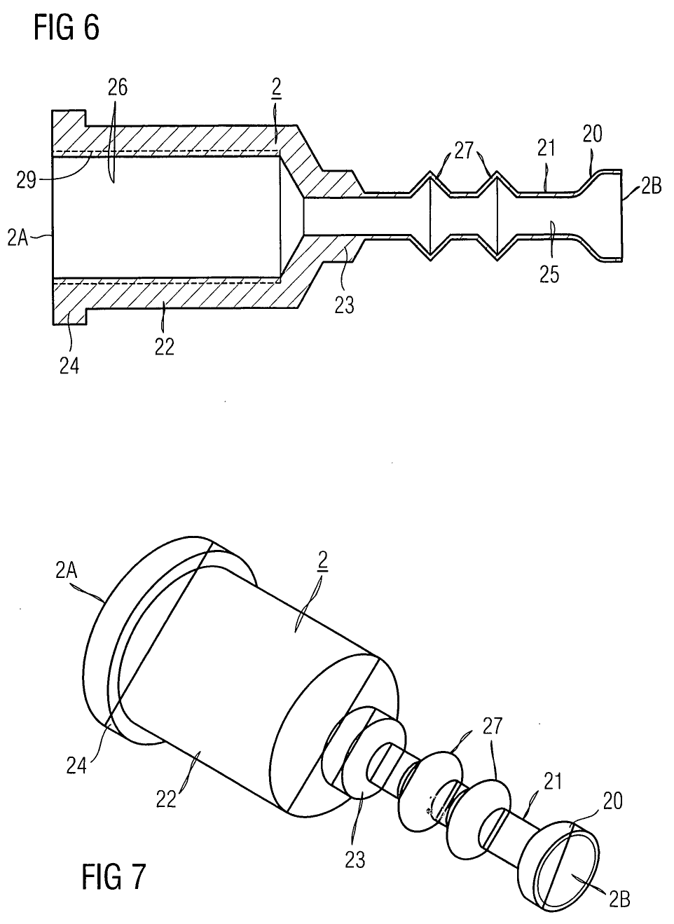

FIG. 6 shows a cross-sectional view of another embodiment of a sealing element, with a contact surface area that has been modified compared to FIG. 4,

FIG. 7 shows the sealing element according to FIG. 6 in a perspective view,

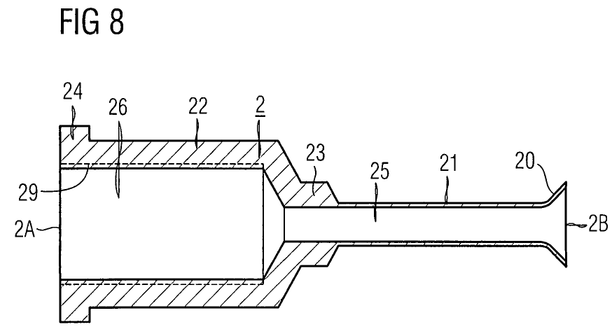

FIG. 8 shows another embodiment of a sealing element in a cross-sectional view,

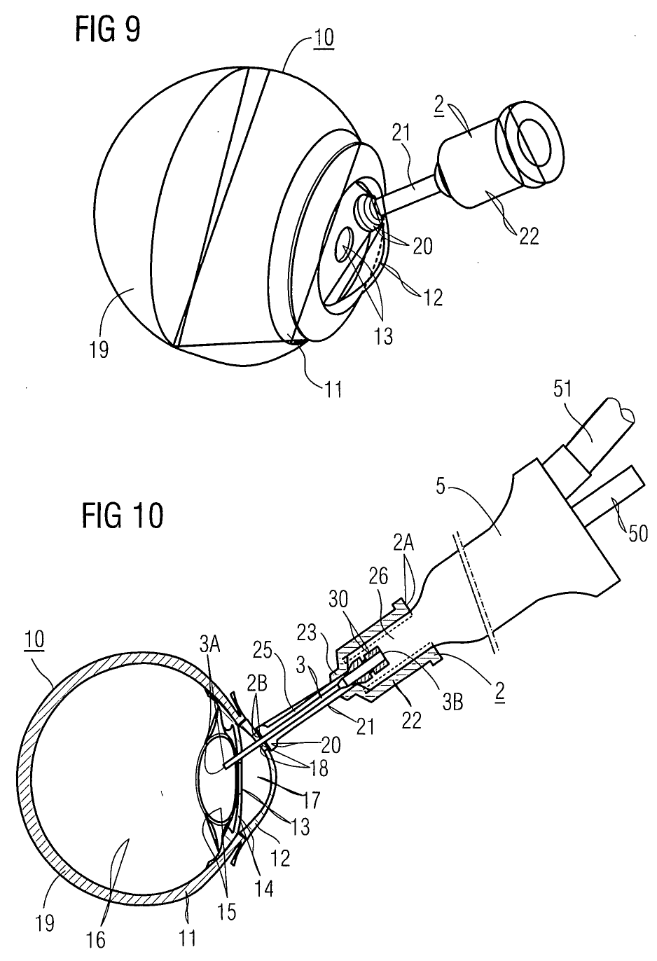

FIG. 9 shows a perspective view of another embodiment of a sealing element with a laser instrument during use on the eye,

FIG. 10 shows the arrangement according to FIG. 9 in a cross-sectional view,

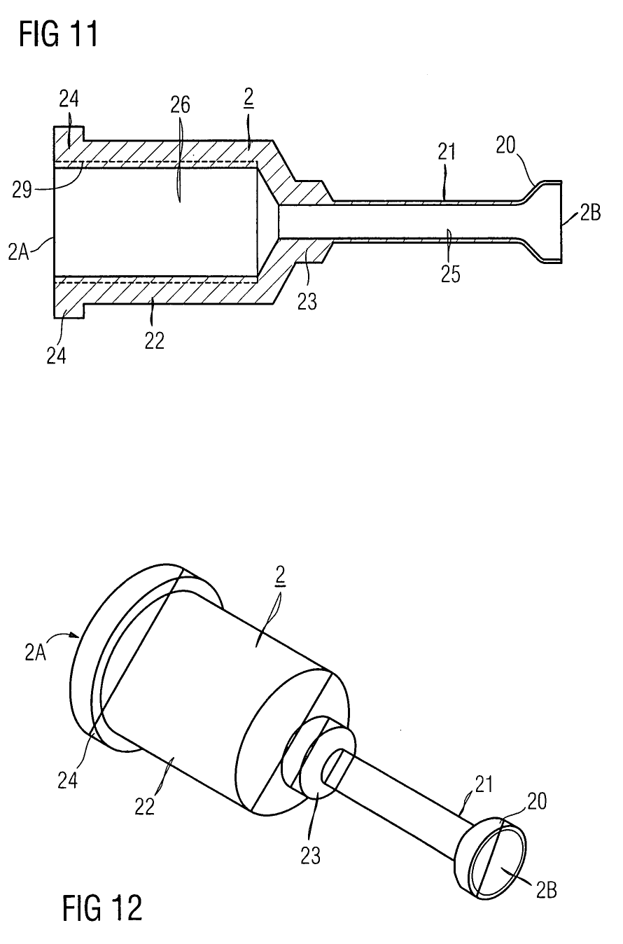

FIG. 11 shows a cross-sectional view of the sealing element used in FIGS. 9 and 10 on its own,

FIG. 12 shows the sealing element according to FIG. 11 in a perspective view,

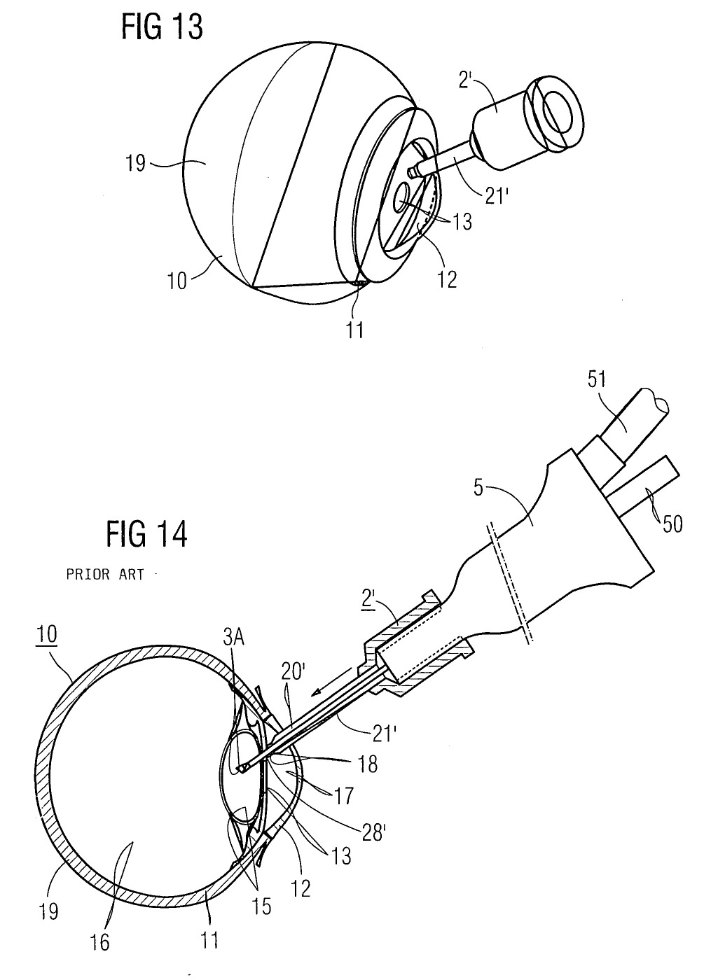

FIG. 13 shows a sealing element designed according to the prior art, during use on the eye,

FIG. 14 shows a cross-sectional view of the arrangement according to the prior art, from FIG. 13,

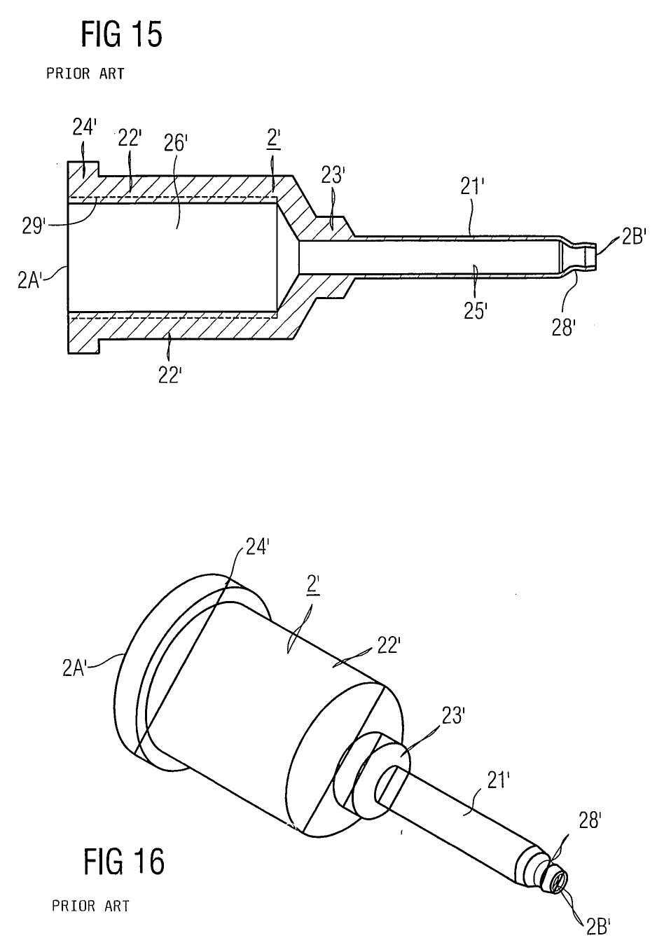

FIG. 15 shows an enlarged cross-sectional view of a sealing element according to the prior art, from FIGS. 13 and 14,

FIG. 16 shows a perspective view of the sealing element according to the prior art, from FIG. 15,

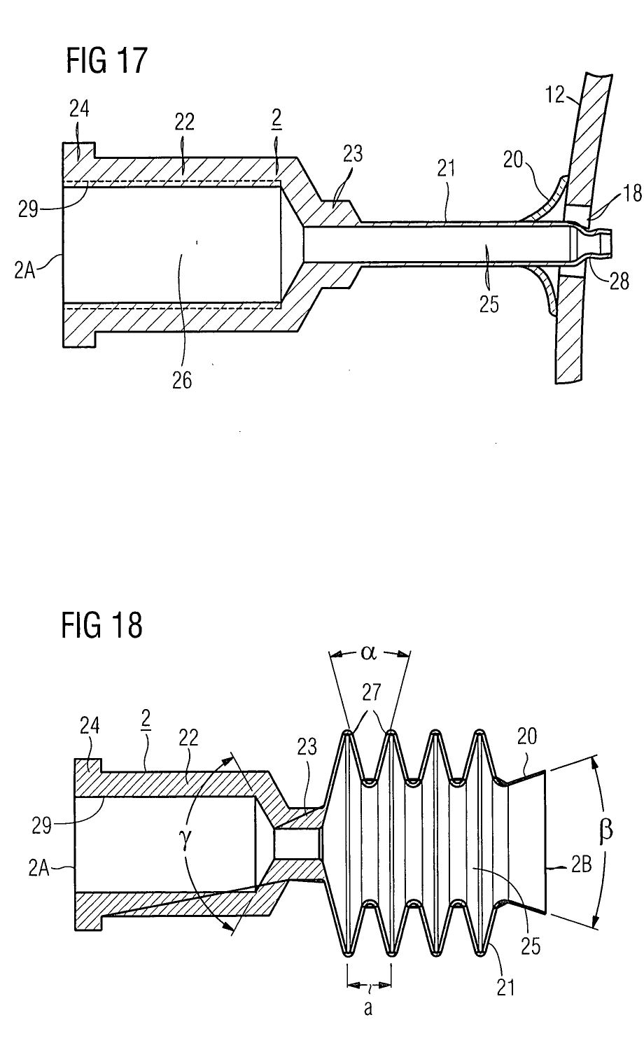

FIG. 17 shows a cross-sectional view of another sealing element according to the invention,

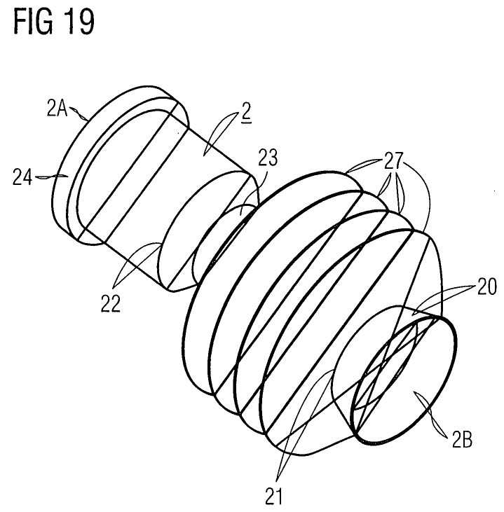

FIG. 18 shows a cross-sectional view of another sealing element, and

FIG. 19 shows the sealing element according to FIG. 18 in a perspective view.

Parts and dimensions corresponding to one another have been provided with the same reference signs in FIGS. 1 to 19.

FIGS. 1 to 3 are schematic views illustrating the use of an operating instrument 3 for lens extraction during a cataract operation.

The eye is designated by 10. The vitreous body 16 and, in front of the vitreous body 16, the crystalline lens 15 are arranged in the eye interior enclosed by the sclera 11, cornea 12 and sclerotic coat 19. The lens 15 is enclosed by the capsular bag (not shown) and is suspended on the sclera 11 via the zonular fibers on the ciliary muscle. The iris 14, which surrounds the pupil 13 and adjusts the latter's size, is arranged in front of the lens 15. Arranged in front of the iris 14 and the pupil 13 is the anterior chamber 17, which is filled with aqueous humor and is delimited anteriorly by the transparent cornea 12.

A cataract operation now generally comprises the following steps:

First, the anterior capsular bag of the lens 15 is opened by means of a surgical instrument, for example a cannula, the opening generally measuring 4.5 mm to 5.5 mm (capsulorhexis). By introducing an irrigation liquid, for example BSS, the lens 15 is released from the capsular bag and thus mobilized (hydrodissection).

In an operating technique using only one instrument (monomanual technique), only one opening (incision) 18 is made in the cornea, particularly at the limbus, preferably at the transition from cornea 12 to sclera 11, and, in another operating technique using two instruments (bimanual technique), two incisions are created, generally on opposite sides of the cornea, particularly at the limbus. In the case involving just one incision, an operating instrument is inserted that comprises integrated irrigation (or delivery of irrigation liquid) and aspiration (or suctioning of tissue and liquid). In the case involving two incisions, an operating instrument with an integrated aspiration system is inserted through one incision, and a separate irrigation instrument for irrigation is inserted through the other incision.

Irrigation liquid, again generally BSS, is then introduced into the capsular bag, and the pressure that is built up in this way ensures that the posterior wall of the capsular bag does not come too close to the operating instrument 3, while at the same time the capsular bag can also be cleaned.

FIGS. 1 to 3 show the operation phase in which an opening 18 has already been created in the outer membrane of the eye for a monomanual technique. In a bimanual technique, a further opening can simply be made on the opposite side, through which further opening the irrigation instrument is then guided.

An operating instrument known per se, and working with ultrasonic oscillations, is guided through the opening 18. It penetrates with its free end 3A into the capsular bag and extracts the lens 15 from said capsular bag. The operating instrument 3 is needle-shaped or cannula-shaped and has an aspiration opening (not shown in detail) at its free end 3A, and an aspiration or suction channel extending from the aspiration opening through the interior of the operating instrument 3.

By way of a handpiece 5 connected or coupled releasably to a widened attachment area at the other end 3B of the operating instrument 3, in particular by means of a piezoelectric oscillation drive (piezo drive) located in the handpiece 5, the operating instrument 3 is driven in oscillations in the ultrasound spectrum, typically above 20 kHz, for example at 40 kHz, and, by means of the energy introduced in this way, destroys the tissue of the lens 15.

The handpiece 5 for its part has an internal aspiration channel that connects the aspiration channel in the operating instrument 3 to an aspiration attachment (or aspiration hose) 50 on the handpiece 5. The aspiration attachment 50 is in turn connected to a suction or delivery device, in particular a pump, for generating an underpressure, typically in the rough vacuum range and/or in the range of an absolute pressure of 700 mbar to 800 mbar. In this way, the destroyed tissue of the lens 15, generally together with liquid located in the capsular bag, is suctioned through the opening at the end 3A of the operating instrument 3, in the suction direction G indicated in FIG. 3, and through the aspiration channels in the operating instrument 3 and in the handpiece 5 and through the aspiration attachment 50.

The handpiece 5 additionally comprises a further attachment 51, which is connected to a cable for delivery of electrical energy for the piezo drive.

The illustrated handpiece 5 thus has an integrated aspiration system.

In one embodiment, the irrigation is performed by means of a separate handpiece. In another embodiment, the handpiece 5 additionally has a further irrigation attachment (not shown) and an internal irrigation channel for delivering irrigation liquid (or flushing and/or cooling liquid), for example BSS. From an irrigation outlet into which the irrigation channel opens, the irrigation liquid then flows out of the handpiece 5 and through the space between the outer wall of the operating instrument 3 guided through the passage 25 and the wall of the sealing element 2 surrounding the passage 25, and then through the opening 18 in the outer area surrounding the operating instrument 3 and into the anterior chamber 17 and into the capsular bag.

This delivery of irrigation liquid serves to compensate for the loss of pressure and loss of substance caused by aspiration in the interior of the eye.

The operating instrument 3 is now arranged in a sealing element 2 and enclosed by the latter. The sealing element 2 comprises a receiving area 22 with a receiving space 26, which is cylindrical, for example, opens out at the end 2A and is surrounded there by an attachment flange 24 that extends farther out than the rest of the wall of the receiving area 22. The receiving area 22 is connected to a spout area 21 via a transition area 23, the diameter decreasing from the receiving area 22 to the spout area 21 by way of the transition area 23, for example in stages. Toward the transition area 23, a conical taper is formed for better flow dynamics. Within the spout area 21 and the transition area 23, a passage 25 is formed, which is cylindrical, for example, and which has a substantially constant diameter smaller than the diameter of the receiving area 26. This passage 25 opens out in a sealing area 20 at the front free end 2B.

The end of the handpiece 5 and the attachment area 30 of the operating instrument 3 are received in the receiving area 26 of the sealing element 2. A thread 29 in the receiving area 26 is for this purpose turned onto an outer thread of the handpiece 5, such that a screwed union is obtained.

The operating instrument 3 (the remaining needle-shaped part) extends through the passage 25 of the sealing element 2 and protrudes with its free end 3A and an adjoining area out of the sealing element 2 and is thus available with its end 3A for the operation.

At least in the area of the spout 21, the sealing element 2 is made of an elastic material, for example of a natural or synthetic rubber or elastomer and/or a thermoplastic elastomer. A particularly suitable material is silicone or silicone rubber (siloxane rubber) or a material with silicone, in particular a vibration-damping material, such as a mixture with elastomer, in particular siloxane elastomer, collagen and water, which is known from WO2004/022999 A1.

To allow the operating surgeon the possibility of identifying where the generally colorless and transparent silicone rubber of the sealing element 2 is lying on the transparent cornea 12, the sealing element 2, in a particular embodiment, can be colored in the area of the sealing area 20.

The wall thickness in the receiving area 22 and in the transition area 23 is preferably much greater than in the spout area 21, such that the spout area 21 is much more deformable or flexible than the receiving area 22. Typical wall thicknesses for receiving area 22 and transition area 23 are greater than 0.7 mm, while the wall thickness of the spout area 21 is chosen typically between 0.2 mm and 0.4 mm.

By means of the concertina-like design of the spout 21 shown in FIGS. 1 to 3, with one, two or more outwardly protruding circumferential folds 27, the spout 21 can be more easily axially compressed.

At the end of the spout 21 of the sealing element 2, at the end 2B of the latter, a sealing area 20 is formed which is widened out in a trumpet shape according to FIGS. 1 to 5 and forms an annular bearing surface as sealing surface at its end.

When the operating surgeon now moves the handpiece 5 with the operating instrument 3 in through the opening 18 toward or into the capsular bag or to the lens 15, he at the same time presses the spout 21 with its sealing area 20 against the outer surface of the cornea 12 and/or sclera 11. The annular sealing surface on the sealing area 20 is spaced apart from the opening 18 and completely surrounds the opening 18. In this way, the sealing area 20 forms a seal, all round the opening 18, against the outer surface of the cornea 12 and/or sclera 11.

By virtue of the elastic deformability or reversible compressibility of the spout 21 under axial compression stress, the operating surgeon can press the spout 21 with sufficient contact pressure against the outer membrane or the eye in order to achieve the necessary pressure-tight sealing of the opening 18. On account of the comparatively large deformation paths of the spout 21 and the resulting relatively great elastic restoring forces in the spout 21 on the one hand, and its spatial distance from the opening 18 on the other hand, the seal is relatively insensitive to a change in position of the operating instrument 3 with the spout 21 both in the lateral direction and also in the axial direction. The sealing surface of the sealing area 20 remains lying fully on the outer membrane of the eye (here 11 or 12) even in the event of changes in shape of the opening 18 that are caused by the movements of the operating instrument 3.

Moreover, in one embodiment with integrated irrigation, the elastic restoring forces mean that the wall of the spout 21 in the sealing area 20 sufficiently counteracts the internal pressure built up in the spout 21 by the irrigation liquid. This therefore reliably ensures that the irrigation liquid flowing through the opening 18 does not leak out around the opening 18.

FIGS. 6 and 7 show a sealing element whose stricture is similar to that of the sealing element according to FIGS. 4 and 5, except that in the sealing area 20 it has a bell-shaped or dome-shaped configuration and not a trumpet-shaped widening. Therefore, whereas the trumpet-shaped sealing area 20 in FIGS. 4 and 5 is curved outward or concavely and continuously increases in diameter toward the end 2B, the sealing area 20 in FIGS. 6 and 7 is curved inward or convexly and increases in diameter with a pitch or rate of increase reducing toward the end 2B, the diameter then remaining constant along a partial area at the end 2B. In FIGS. 6 and 7, therefore, the sealing area 20 is designed like a plunger. Many other shapes of the sealing area 20 are of course also possible, for example bladder shape or the shape of sealing lips, etc.

FIG. 8 shows another embodiment of a sealing element 2 in which, in contrast to FIGS. 4 and 5, the two folds 27 are omitted.

FIGS. 9 and 10 show a sealing element 2 which is derived from the sealing element according to FIGS. 6 and 7 through omission of the folds 27. The sealing element 2 is placed on the eye, and its sealing area 20 surrounds the opening 18 in order to seal off the eye interior from the outer environment.

The operating instrument 3 in FIGS. 9 and 10 is a laser instrument known per se, for example the aforementioned laser handpiece from A. R. C. Laser GmbH, or an instrument which is constructed in accordance with aforementioned patents U.S. Pat. No. 5,324,282 A or U.S. Pat. No. 5,906,611 A and in which the crystalline lens is photolytically broken up successively by targeted laser pulses (e.g. pulse duration of 2 to 10 ns, up to 20 pulses per second) and the resulting shockwaves and is then suctioned out.

FIGS. 11 and 12 show the sealing element 2 used in FIGS. 9 and 10, in a more detailed representation.

FIGS. 13 and 14 show a monomanual technique with an ultrasonic operating instrument 3 which is surrounded by a sealing element 2′ according to the prior art, also called a sleeve, for protecting the surrounding eye tissue from the vibrating operating instrument 3. The known sealing element 2′ is shown enlarged in FIGS. 15 and 16 and differs from the inventive sealing elements 2 shown in FIGS. 1 to 10 in terms of the configuration of the sealing area compared to the sealing area 20 in the invention. In the remaining areas, the reference signs are simply provided with a prime mark in order to illustrate the correspondence. The passage 25′ in the spout area 21′ of the sealing element 2′ opens out in the end area 28′ at the front free end 2B′. In the end area 28′, the diameter of the spout area 21′ in the prior art now tapers toward the end 2B′ in the manner of a glass bottle top, where outlet openings for the irrigation liquid S can be present. The known sealing element 2′ is inserted with the cylindrical outer face of the spout area 21′ through the opening 18, and the tapering end area 28′ serves as an insertion aid.

According to the invention, and in contrast to the prior art according to FIGS. 13 to 16, the opening 18 in the cornea 12 and/or sclera 11 is not sealed (exclusively) on the inner face or the surrounding edge of the opening 18, but on the outer face of the sclera 11 and/or cornea 12 by means of a sealing surface that completely surrounds the opening 18.

FIG. 17 shows another embodiment of a sealing element 2 according to the invention, in which the sealing area 20 is not arranged at the end 2B of the sealing element 2, but on the outer wall of the spout area 21, in the manner of a circumferential sealing lip. At the end 2B, the sealing element 2 is designed like the known sealing element 2′, in other words has the tapering end area 28. Therefore, in the sealing element 2 according to FIG. 17, a seal can be formed by means of the sealing area 20 around the opening 18 on the outer surface of the cornea 11 in the area 12, and a seal can also be formed in the opening 18 by the end area 28 or the outer surface of the spout area 21.

In the other embodiment of a sealing element 2 according to FIGS. 18 and 19, a particularly flexible concertina is provided in the area of the spout 21. The spout 21 has four circumferential folds 27 that are spaced apart by a distance a and whose flank areas enclose an angle α. The spacing a of the folds 27 can lie in a range between 0.5 mm and 3 mm and 5 mm, in particular about 2.5 mm. The aperture angle α can lie between 20° and 60°, in particular 30°. The wall thickness of the spout 21, at least in the area of the folds 27, can be chosen between 0.1 mm and 0.5 mm, in particular approximately 0.2 mm. Toward the free end 2B, the spout 21 runs out in a sealing area 20, which widens in a funnel shape or conically outward or toward the end 2B at an aperture angle β, which is typically between 20° and 60°, for example between 30° and 40°. The wall thickness of the spout 21 in the sealing area 20 is preferably smaller than in the area of the folds 27 and can, for example, be in a range of between 0.05 mm and 0.3 mm, in particular approximately 0.10 to 0.15 mm. In this way, the sealing area 20 is still particularly flexible and can bear very gently on the surface of the cornea 12 and/or sclera 11. FIG. 18 also indicates the aperture angle γ of the conically tapering transition area 23, which angle, in the illustrative embodiment shown, is 1200, although it can also deviate from this value.

After complete removal of the natural lens and cleaning of the capsular bag, the cataract operation is concluded by inserting an artificial lens into the capsular bag and then closing the wounds.

### LIST OF REFERENCE SIGNS

2, 2′ sealing element
2A, 2B end
3 operating instrument
3A, 3B end
5 handpiece
10 eye
11 sclera
12 cornea
13 pupil
14 iris
15 lens (crystalline lens)
16 vitreous body
17 anterior chamber of the eye
18 opening
19 sclerotic coat
20, 20′ sealing area
21, 21′ spout
22, 22′ receiving area
23, 23′ transition area
24, 24′ attachment flange
25, 25′ passage
27 fold
28, 28′ sealing area
29, 29′ thread
30 attachment area
50 irrigation attachment
51 attachment
α, β, γ angles

1. A device for sealing an opening in a human or animal eye, comprising:
at least one sealing element comprising:
a) with a passage surrounded by a wall,
b) at least one sealing area having a sealing, surface constructed to-be placed on an outer surface of the eye surrounding the opening wherein sealing surface completely surrounds the opening, and wherein
an operating instrument can be passed into an interior of the eye through the passage in the sealing element and through the opening.

2. The device of claim 1, wherein the opening in the eve comprises a surgically created opening in one or more than one membrane of the eve selected from among cornea, sclera, and sclerotic coat.

3. The device of claim 1, wherein the sealing element is elastically deformable at least in the sealing area.

4. The device of claim 1, wherein the sealing surface of the sealing area in an undeformed state is substantially ring-shaped.

5. The device of claim 1, wherein the sealing surface of the sealing area in an undeformed state is substantially flat.

6. The device of claim 1, in which the sealing surface of the sealing area in the undeformed state comprises a substantially curved shape to match a curvature of the eye.

7. The device of claim 1, wherein the sealing area widens toward the sealing surface.

8. The device of claim 7, wherein the sealing area widens toward the sealing surface with a shape selected from among the shapes of a concavely shape, a funnel shape, and a trumpet shape convex shape, a bell shape, and a dome shape.

9. The device of claim 1, wherein at least one dimension of the sealing surface in a direction selected from a direction toward the opening, a radial direction and a direction of a thickness of the wall in the sealing area is chosen in a range between 0.1 mm and 7 mm.

10. The device of claim 1, wherein a dimension of the sealing surface in at least a direction selected from a direction toward the opening, a radial direction and a thickness direction of the wall in the sealing area is chosen in a range between 0.2 mm and 1.5 mm.

11. The device of claim 1, wherein a maximum diameter of the outer surface of the eye surrounded by the sealing surface is between approximately 3 mm and 10 mm.

12. The device of claim 1, wherein a maximum diameter of the opening in the eye is between approximately 1.5 mm and approximately 6 mm.

13. The device of claim 1, wherein the sealing surface of the sealing area of the sealing element placed on the surface of the eye is spaced apart from the opening in the eye, by a distance from about 0.1 mm to about 2 mm.

14. The device of claim 1, wherein the minimum diameter of the surface of the eye surrounded the sealing surface is greater than the maximum diameter of the opening by about 0.1 mm to 2 mm.

15. The device in claim 1, wherein the passage in the sealing element has a first passage area and a second passage area adjoining the first passage area, the first passage area at least partially forming a receiving space for receiving one or more of a part of the operating instrument and a handpiece for the operating instrument, and wherein the second passage area and the sealing area are configured to guide the operating instrument therethrough.

16. The device as claimed in claim 15, wherein the first passage area has a larger internal cross section than the second passage area.

17. The device as claimed in claim 15, wherein the wall of the first passage area is thicker than the wall of the second passage area.

18. The device as claimed in one of claims 15 wherein the sealing element comprises a spout area and wherein the second passage area extends into the spout area.

19. The device as claimed in one of claims 15 which the wall of the second passage area or the spout area has at least one circumferential fold or is designed like a concertina.

20. The device of claim 1, wherein the passage opens out in the sealing area.

21. The device of claim, 1, wherein the passage opens out in a mouth area, and the sealing area is formed on the wall of the passage, and comprises a spout area having a circumferential sealing lip.

22. The device as claimed in claim 21, wherein, a second sealing area having a second sealing surface is provided in the mouth area of the passage and bears against outer tissue on an inner edge surface of the opening in the eye.

23. The device as of claim 1, wherein the sealing element is constructed in one piece comprising a shaped body made from one material.

24. The device of claim 1, wherein the sealing element is at least partially composed of an elastic material, selected from one or more of on a natural rubber, a synthetic rubbers, an elastomer, or a siloxane rubber.

25. The device of claim 1, wherein the sealing surface of the sealing, area is colored or is designed to have minimal optical transparency or to be non-transparent.

26. The device Of claim 1, further comprising irrigation means for conveying irrigation liquid through the passage of the sealing element, via a space between the operating instrument and the sealing element, and through the opening into an interior of the eye.

27. The device of claim 26, wherein the irrigation means are integrated in a handpiece configured to attach to the operating instrument.

28. The device of claim 1, wherein the operating instrument is elongate, having a shape selected from a needle shape or a cannula shape, and having a maximum external diameter of 3 mm.

29. The device of claim 1, wherein the operating instrument comprises one or more instruments selected from an ultrasonic operating instrument, a photolysis instrument, an operating instrument for phacolysis, a cutting instrument, a scalpel instrument, an operating instrument for vitrecotomy, an irrigation instrument, and a manipulation instrument.

30. (canceled)

31. (canceled)

32. (canceled)

33. (canceled)
